(19) United States
(12) Patent Application Publication
(10) Pub. No.: US 2020/0234801 A1
(43) Pub. Date: Jul. 23, 2020

(54) METHODS AND SYSTEMS FOR HEALTHCARE CLINICAL TRIALS

(71) Applicant: KONINKLIJKE PHILIPS N.V., EINDHOVEN (NL)

(72) Inventors: Yong Mao, Hawthorne, NY (US); Woei-Jye Yee, Boston, MA (US); Alexander Ryan Mankovich, Somerville, MA (US); Qingxin Wu, Lexington, MA (US); Kostyantyn Volyanskyy, Larchmont, NY (US); Nevenka Dimitrova, Pelham Manor, NY (US)

(21) Appl. No.: 16/652,878

(22) PCT Filed: Oct. 5, 2018
(86) PCT No.: PCT/EP2018/077139

§ 371 Date: Apr. 1, 2020

Related U.S. Application Data
(60) Provisional application No. 62/732,651, filed on Sep. 18, 2018.
(60) Provisional application No. 62/568,884, filed on Oct. 6, 2017.

(51) Int. Cl. G16H 10/20; G16H 10/60

(52) U.S. Cl. CPC G16H 10/20; G16H 10/60

(57) ABSTRACT

A method (100) for recruiting a patient for a clinical trial, comprising: receiving (110) a dataset comprising information about one or more clinical trials each including patient eligibility criteria; extracting (120) the patient eligibility criteria from each of the clinical trials; converting (130) the patient eligibility criteria to a standardized patient eligibility criterion using a structured clinical trial mark-up language; storing (140) the patient eligibility criterion in a database (862), each of the criterion associated with one or more clinical trials; receiving (150) patient-specific data values about a patient; querying (160) the clinical trial eligibility criteria database using the patient-specific data values to identify eligibility criterion satisfied by the patient-specific data value; identifying (170) a clinical trial associated with the one or more standardized patient eligibility criterion satisfied by a received patient-specific data value; and providing (180) a report of the identification of the at least one clinical trial.

### CROSS-REFERENCE TO RELATED APPLICATIONS

This application claims priority to U.S. Provisional Patent Application Ser. Nos. 62/568,884, filed on Oct. 6, 2017, and 62/732,651, filed on Sep. 18, 2018, both entitled “METHODS AND SYSTEMS FOR HEALTHCARE CLINICAL TRIALS,” the entire contents of which are incorporated herein by reference.

### FIELD OF THE DISCLOSURE

The present disclosure is directed generally to methods and systems for recruiting patients for a clinical trial.

### BACKGROUND

Clinical trials, conducted under specific healthcare protocols, are of vital importance in the treatment of many diseases. Unfortunately, a clinical trial if a sufficient number of eligible patients are not enrolled in a reasonable time. Additionally, there are significant barriers to identifying clinical trials and matching patients with those clinical trials. This can be especially significant for late-stage cancer patients, among others, where there is an urgency to identify a matching clinical trial.

Current clinical trial matching methods and systems are based on keyword matching systems, which match a query string to key words found within or extracted from a clinical trial document. However, keywords are not able to describe or accurately identify the features and criteria needed for clinical trial patient matching. Indeed, conventional clinical trial matching systems do not possess the specificity and precision required to search and identify clinical trials due to the many drawbacks of keyword searching. Accordingly, keyword searching does not perform the matching necessary to identify patient-specific clinical trials, and thus current solutions are inadequate for using patient-specific data to automatically compare with clinical trial documents and identify pertinent patient specific data and criteria to recruit patients to clinical trials.

### SUMMARY OF THE DISCLOSURE

There is a continued need for methods and systems that match a patient with a clinical trial using a specialized markup language for clinical trial information. Various embodiments and implementations herein are directed to a method and system configured to recruit patients for clinical trials using a clinical trial matching system. The system receives a dataset comprising information about clinical trials, each clinical trial including patient eligibility criteria. The system extracts the patient eligibility criteria and converts them to standardized patient eligibility criteria using a structured clinical trial mark-up language. The standardized patient eligibility criteria, each associated with the respective clinical trial, are then stored in a searchable clinical trial eligibility criteria database. To match patients with clinical trials, the system receives patient-specific data values about a patient and the clinical trial eligibility criteria database is queried using these data values to identify one or more standardized patient eligibility criterion satisfied by a received patient-specific data value. One or more clinical trials suitable for the patient are identified based on standardized patient eligibility criteria for that clinical trial being satisfied by the patient-specific data value. The system then provides a report of the one or more identified clinical trials suitable for the patient, which may optionally be ranked based on how many eligibility criteria the patient satisfies.

Various embodiments relate to a clinical trial markup language for addressing the information structuralization problem in clinical trial data recording and storage for recruiting patients to clinical trials. Among other things, the information that is structuralized from clinical trials includes eligibility criteria. According to an embodiment, the clinical trial markup language defines international vocabularies incorporating medical terms and/or Unified Medical Language features, as well as expression logic, to translate unstructured clinical trial documents into a computable format. The system can provide increased speed and accuracy for clinical trial patient matching, thus greatly benefiting medical research, clinical trials, patients, and overcoming the additional problem of the lack of interoperability between clinical trial documents and patient clinical data residing in medical records.

The system and method can be used for providing efficient recruitment of patients for clinical trials. A method is described for providing interoperability between clinical trial document and patient clinical data residing in medical records. The method includes steps for providing a dataset of textual documents from a clinical trial, the documents containing obscured and non-obscured patient eligibility criteria, storing the documents on a server, formatting the documents in a natural language with patient eligibility criteria, translating the formatted patient eligibility criteria into a series of structured query language queries, inputting patient-specific data values, performing at least one query search of the patient eligibility criteria, and recruiting at least one patient for the clinical trial so that at least one patient-specific data value matches a patient eligibility criteria of the clinical trial, as well as displaying a list of patients matched to the clinical trial. Various embodiments provide a system and method for providing a list of pertinent patient-specific clinical trials based on selected searching, structuralizing and matching criteria selected by a user of the system and method.

Generally, in one aspect, a method for matching a patient with a clinical trial using a clinical trial matching system is provided. The method includes: (i) receiving a dataset comprising information about one or more clinical trials, the information comprising one or more patient eligibility criterion for each of the one or more clinical trials; (ii) extracting, by a processor of the system, the one or more patient eligibility criterion from each of the one or more clinical trials; (iii) converting, by the processor, each of the extracted patient eligibility criterion to a standardized patient eligibility criterion using a structured clinical trial mark-up language; (iv) storing the standardized patient eligibility criterion in a searchable clinical trial eligibility criteria database, each of the standardized patient eligibility criterion associated with at least one of the one or more clinical trials; (v) receiving one or more patient-specific data values about a patient; (vi) querying, by the processor, the clinical trial eligibility criteria database using the received one or more patient-specific data values to identify one or more standardized patient eligibility criterion satisfied by a received patient-specific data value; (vii) identifying at least one of the one or more clinical trials, the at least one clinical trial associated with the one or more standardized patient eligibility criterion satisfied by a received patient-specific data value; and (viii) providing a report of the identification of the at least one clinical trial.

According to an embodiment, the method includes ranking two or more identified clinical trials, wherein the ranking is based at least in part on a number of standardized patient eligibility criterion satisfied by received patient-specific data values, and wherein the report comprises information about the ranking of the two or more identified clinical trials.

According to an embodiment, the report is provided via a user interface of the system.

According to an embodiment, the dataset comprising information about one or more clinical trials is comprised of information from a plurality of sources.

According to an embodiment, the step of converting the extracted patient eligibility criterion to a standardized patient eligibility criterion comprises a machine learning algorithm.

According to an embodiment, the step of converting the extracted patient eligibility criterion to a standardized patient eligibility criterion comprises resolving a complex eligibility criterion into one or more simple eligibility criteria. According to an embodiment, the one or more simple eligibility criteria are joined by one or more Boolean operators.

According to an embodiment, the one or more patient eligibility criterion comprise inclusion criteria and exclusion criteria.

According to an embodiment, the one or more patient-specific data values are obtained from a patient medical record.

According to an aspect is a system for matching a patient with a clinical trial. The system includes: a clinical trial eligibility criteria database comprising information about a plurality of clinical trials, each of the plurality of clinical trials comprising one or more patient eligibility criterion; and a processor configured to: (i) extract the one or more patient eligibility criterion from each of the one or more clinical trials; (ii) convert each of the extracted patient eligibility criterion to a standardized patient eligibility criterion using a structured clinical trial mark-up language; (iii) store the standardized patient eligibility criterion in the clinical trial eligibility criteria database, each of the standardized patient eligibility criterion associated with at least one of the one or more clinical trials; (iv) receive one or more patient-specific data values about a patient; (v) query the clinical trial eligibility criteria database using the received one or more patient-specific data values to identify one or more standardized patient eligibility criterion satisfied by a received patient-specific data value; (vi) identify at least one of the one or more clinical trials, the at least one clinical trial associated with the one or more standardized patient eligibility criterion satisfied by a received patient-specific data value; and (vii) generate a report of the identification of the at least one clinical trial.

According to an embodiment, the system includes a patient information database, the patient information database comprising one or more patient-specific data values.

According to an aspect is a method for recruiting one or more patients for a clinical trial using a clinical trial matching system. The method includes: (i) receiving a dataset comprising information about one or more clinical trials, the information comprising one or more patient eligibility criterion for each of the one or more clinical trials; (ii) extracting, by a processor of the system, the one or more patient eligibility criterion from each of the one or more clinical trials; (iii) converting, by the processor, each of the extracted patient eligibility criterion to a standardized patient eligibility criterion using a structured clinical trial mark-up language; (iv) receiving one or more patient-specific data values about a patient, and storing the patient-specific data values in a patient information database; (v) querying, by the processor, the patient information database using the standardized one or more patient eligibility criterion to identify one or more patients eligible for a clinical trial; (vi) identifying at least one of the patients, the at least one patient associated with a patient-specific data value satisfying a standardized patient eligibility criterion used to query the patient information database; and (vii) providing a report of the identification of the at least one patient.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. The figures showing features and ways of implementing various embodiments and are not to be construed as being limiting to other possible embodiments falling within the scope of the attached claims. Also, the drawings are not necessarily to scale, emphasis instead generally being placed upon illustrating the principles of the various embodiments.

FIG. 1 is a method for matching a patient(s) with a clinical trial(s), in accordance with an embodiment.

FIG. 2 is a flowchart of clinical trial recruitment and matching methods, in accordance with an embodiment.

FIG. 3 is a flowchart of an analyzer pipeline, in accordance with an embodiment.

FIG. 4 is an example of a semantic network to mark up from clinical trial eligibility criteria, in accordance with an embodiment.

FIG. 5 is a block diagram of a method for ranking, in accordance with an embodiment.

FIG. 6 is an embodiment of a GUI web application, in accordance with an embodiment.

FIG. 7 is a flowchart of clinical trial recruitment and matching methods, in accordance with an embodiment.

FIG. 8 is a schematic representation of a system for matching a patient(s) with a clinical trial(s), in accordance with an embodiment.

### DETAILED DESCRIPTION OF EMBODIMENTS

The present disclosure describes various embodiments of a system and method configured to match a patient with a suitable clinical trial. More generally, Applicant has recognized and appreciated that it would be beneficial to provide a system that more accurately and more efficiently identifies clinical trials for which a patient is eligible. The system receives information about clinical trials, each clinical trial including one or more patient eligibility criteria. The system extracts the patient eligibility criteria from the clinical trials and converts them to standardized patient eligibility criteria using a structured clinical trial mark-up language. The standardized patient eligibility criteria, each associated with the respective clinical trial from which they were extracted, are then stored in a searchable clinical trial eligibility criteria database. To match patients with clinical trials, the system receives patient-specific data values about a patient and the clinical trial eligibility criteria database is queried using these data values to identify one or more standardized patient eligibility criterion satisfied by a received patient-specific data value. One or more clinical trials suitable for the patient are identified based on standardized patient eligibility criteria for that clinical trial being satisfied by the patient-specific data value. The system then provides a report of the one or more identified clinical trials suitable for the patient, which may optionally be ranked based on how many eligibility criteria the patient satisfies.

In certain embodiments, patients, families, physicians and medical researchers can identify promising trials that may benefit a particular patient. By entering trial information into the public repository following structured clinical trial mark-up language modalities and definitions, and by using natural language processing tools specifically designed to translate trial descriptions into the structured clinical trial mark-up language, the performance in speed and accuracy of clinical trial matching and recruitment can be dramatically improved, greatly benefiting both medical research and patients.

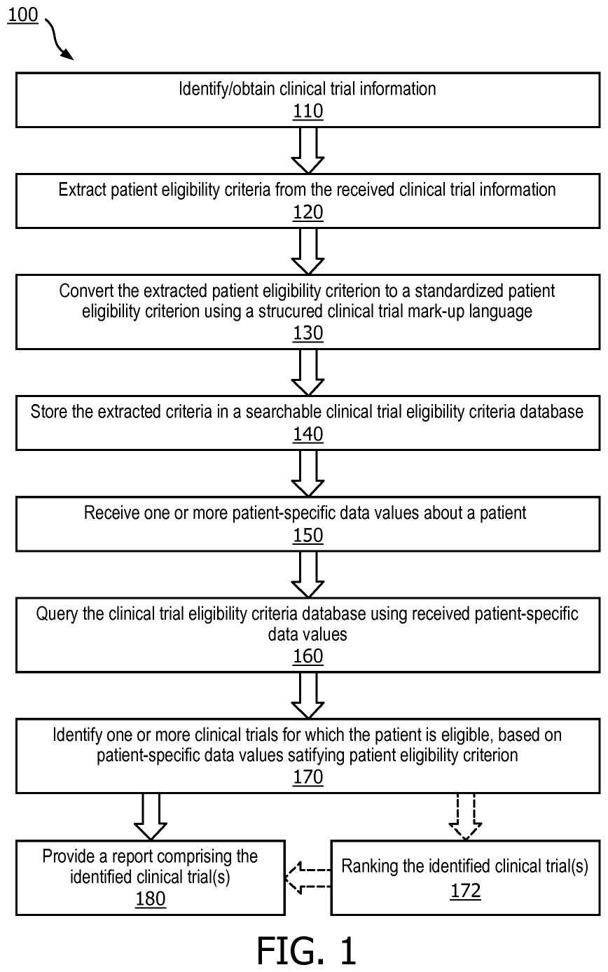

Referring to FIG. 1, in one embodiment, is a flowchart of a method 100 for identifying a clinical trial for which a patient is eligible using a clinical trial matching system. The methods described in connection with the figures are provided as examples only, and shall be understood not to limit the scope of the disclosure. The clinical trial matching system can be any of the systems described or otherwise envisioned herein.

At step 110 of the method, one or more clinical trial documents or other clinical trial sources are obtained or received by the clinical trial matching system. These clinical trial documents or other sources can be any text, document, or other record or source comprising text or images about a clinical trial. According to a preferred embodiment, the clinical trial information comprises digital or digitized documents, and may be obtained from one or more different sources of such clinical information. For example, among other sources, the clinical trial information may be obtained or received from government clinical trial sources, NIH sources, NCBI sources, clinical trial registries, institutional review board (IRB) documents, independent ethics committee (IEC) documents, ethical review board (ERB) documents, research ethics board (REB) documents, online clinical trial registries, self-service clinical trial registries, international clinical trial sources, private sources, hospitals, medical research institutes, EudraCT, ClinicalTrials.gov, Drugs@ FDA FDA1572, YODA, PubMed, The Sunshine Act Database, and WHO, and/or UMIN, among many other possible sources. These are just examples and not meant to be exhaustive. According to an embodiment, the documents comprise clinical summary documents. According to an embodiment, the clinical trial documents or other clinical trial sources comprise a Health Level Seven International (HL7) format, among many other possible formats.

According to an embodiment, a clinical trial document may generally follow FDA requirements for recordkeeping and record retention for clinical research contained in 21 CFR 312.62 and 812.140, which cover disposition of study drug and experimental devices, case histories, and record retention. Case histories may contain information concerning aspects of the trial investigation, as well as case report forms and supporting data. Supporting data can be source data and may be contained in source documents. Clinical trial document can comprise information based on the International Committee on Harmonization' E6 consolidation guide for GCP definitions. Source data can be information in original records and certified copies of original records or clinical findings, observations, or other activities in a clinical trial necessary for the reconstruction and evaluation of the trial. Source data may be contained in source documents, such as original records and/or certified copies. Examples of source documents include original documents, data and records (e.g., hospital records, clinical and office charts, laboratory notes, memoranda, subjects' diaries, pharmacy dispensing records, recorded data from automated instruments, transcriptions, microfiches, photographic negatives, microfilm, magnetic media, x-rays, pharmacy records, and medical department records involved in the clinical trial. In some aspects, source data may be in case report forms.

The sources can be provided to the clinical trial matching system by an individual or another system. Additionally and/or alternatively, the sources can be retrieved by the clinical trial matching system. For example, the clinical trial matching system may continuously or periodically access any database, website, or any other resource comprising or providing clinical trial information. As just one example, the clinical trial matching system may automatically access any of the sources listed or envisioned above. As just one example, a continuous stream of incoming clinical trials from, e.g., clinicaltrials.gov, as well as other sources, may be regularly maintained so that the database can be constantly updated with new clinical trial information.

The received or obtained clinical trial documents or other clinical trial sources may be stored in a local or remote database for use by the clinical trial matching system. For example, a clinical trial can be stored as an xml file on a local server. The clinical trial matching system may comprise a database to store the clinical trial information, and/or may be in communication with a database storing the information. These databases may be located with the clinical trial matching system or may be located remote from the clinical trial matching system, such as in cloud storage and/or other remote storage.

An eligibility criterion may be any criterion that must be satisfied by a patient for eligibility in a clinical trial. For example, patient eligibility criteria comprise inclusion criteria, which are criteria that the patient must meet to be included, and exclusion criteria, which are criteria that would exclude the patient from inclusion in the clinical trial. Among many other criteria, the eligibility criteria may comprise age, gender, disease type, disease stage, previous treatment history, other medical conditions, location, manifestation, symptom, sign, lab test results, sign symbols, sign threshold, temporal constraint, body location, diagnosis, assessment, medical specialty, device, consequence of condition, stage of condition or disease, grade of lesion or tumor, therapy, surgery, medication, dosage, mechanism of action, medication form, consent, enrollment in other studies, demographics, literacy, spoken language, lifestyle, and/or addictive behavior, among many other possible eligibility criteria.

A criterion may be simple or complex. A simple criterion may consist of, for example, a single noun phrase (menopausal), its negation (no hypertension), or a simple quantitative comparison (age>=18 years). Complex criteria typically vary in content, the use of negation, Boolean connectors, arithmetic comparison operators, temporal connectors, comparison operators, if-then constructions, and/or a combination of all of the above, among other possibilities.

At step 120 of the method, the clinical trial matching system extracts the patient eligibility criteria from each clinical trial. The eligibility criteria may be identified and/or extracted using any of a number of possible mechanisms. According to an embodiment, the clinical trial matching system comprises a language analyzer or other algorithm, such as a machine learning algorithm, configured to identify an eligibility criterion and extract or otherwise isolate or characterize the identified eligibility criterion for downstream processing or analysis by the system. According to another embodiment, a user identifies and/or extracts eligibility criteria from the clinical trial document or source.

According to an embodiment, a clinical trial document may be prepared for extraction, by either a user or a system, by eliminating vague descriptions and/or redundant or unnecessary language from the description, and/or by compound eligibility criteria into stand-alone eligibility criteria. Standardizing or normalizing the format of a clinical trial document can facilitate the extraction of eligibility criteria from each clinical trial.

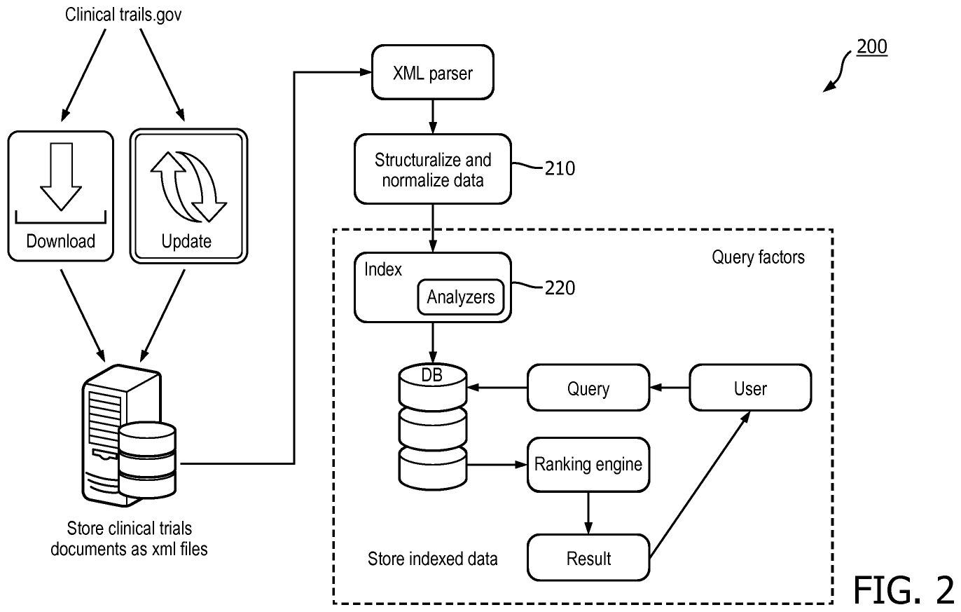

Referring to FIG. 2, in one embodiment, is a flowchart of a method 200 for querying a clinical trial criteria database to identify one or more clinical trials for which a patient is eligible. According to this embodiment, the system receives information about clinical trials and stores the clinical trial information as one or more XML files on a local or remote server. The clinical trial data can be structured and/or normalized using an XML parser 210. The XML document parser may be used to parse the stored clinical trial documents and extract useful information, such as the clinical trial design, eligibility criteria and geographical/location details. In certain embodiments, criteria may be normalized in seconds or minutes.

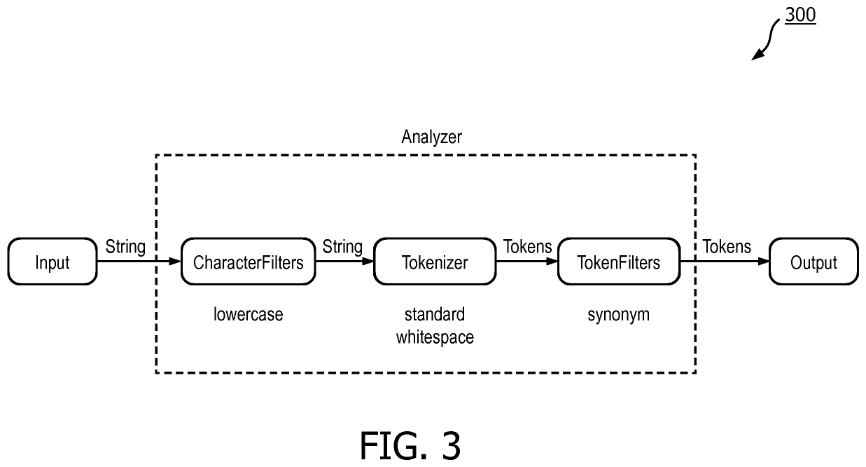

According to an embodiment, the parsed data, which is now structured and/or normalized, can be indexed by an indexer 220 in preparation for storage. Referring to FIG. 3, in one embodiment, is an analyzer process 300 used by indexer 220. For each section of a clinical trial document, strings are first lowercased and tokenized using a built-in tokenizer and whitespace tokenizer. Then, any gene terms, which are often found in the clinical trial document eligibility criteria section, can be passed through a synonym filter where the canonical expression is returned. Because synonyms are very common for genes, incorporating synonyms into the analyzer tool significantly increases the number of potential matches of clinical trials. Gene synonyms such as family names, aliases, previous names, and previous symbols may be obtained from public databases. Similarly, a synonym filter for the disease name can be incorporated to further improve the performance of the trial matching engine when queries also involve disease diagnosis.

In some embodiments an inverted index can be used, which can allow fast, full-text index and query, for full-text searching. An inverted index may consists of a list of all the unique words that appear in any document, and is an index data structure storing a mapping from content, such as words or numbers, to their locations in a document or a set of documents. It is named in contrast to Forward Index, which maps from document to content. For example:

‘hello’: doc1:1, doc3:10 (docid: position)

‘world’: doc1, doc2, doc3 (docid)

For each word, via the hash table or the index there is found a list of the documents in which the word appears. This mechanism can allow faster searching than matching each term in each document.

The indexed, structured, and/or normalized information from the clinical trials, including one or more eligibility criteria, can then be stored for downstream analysis, and/or can be analyzed immediately, as described in greater detail herein.

At step 130 of the method, the extracted patient eligibility criteria are converted to standardized patient eligibility criteria using a structured clinical trial mark-up language (CTML). The CTML, which enables interoperability between one or more clinical trial documents and various patient specific clinical data, can be utilized by one or more natural language processing (NLP) tools such that the clinical trial matching system can convert unstructured clinical trial descriptions into standardized patient eligibility criteria using the CTML. The unique CTML converts obscured and non-obscured patient eligibility criteria from clinical trial information into a standardized format. By capturing both obscured and non-obscured patient eligibility criteria from clinical trial information, a method can provide surprisingly improved speed and/or accuracy for matching and recruitment of patients to clinical trials.

According to an embodiment, natural language processing tools can be used to translate trial information and formatted patient eligibility criteria into a series of structured data suitable for query language (SQL) queries. Examples of NPL tools include but are not limited Stanford's Core NLP Suite, Natural language Toolkit, Apache Lucene and Solr, Apache OpenNLP, GATE, and Apache UIMA, among many other possibilities. In some aspects the natural language may comprise machine learning. For example, clinical trial documents may be tagged for various features, such as parts of speech, persons, institutions, subject matter, or classifiers. The tagged documents can be used for training, and the learned set can be applied to new documents. Among other factors, the system may comprise character recognition and may segment the text of a document as necessary.

According to an embodiment, the unique CTML captures logical relationships between features and terms of a Unified Medical Language System (UMLS), and/or features and terms from clinical trial information. The logical relationships can be captured using Boolean connectors, arithmetic comparison operators, temporal connectors, comparison operators, if-then constructions, or any combination of the foregoing. In certain embodiments, the concepts and relationships captured by the CTML can involve any one or more of location, gender, age, medical condition, manifestation, symptom, sign, lab test results, sign symbols, sign threshold, temporal constraint, body location, diagnosis, assessment, medical specialty, device, consequence of condition, stage of condition or disease, grade of lesion or tumor, therapy, surgery, medication, dosage, mechanism of action, and medication form.

In certain embodiments, the clinical trial matching system resolves the eligibility criteria into single components. The natural language processing may resolve the eligibility criteria into components joined by Boolean operators. In certain embodiments, the natural language processing may tag parts of speech. Due to the complexity of clinical trial design, a trial description may involve eligibility criteria for multiple arms. An eligibility criterion for each arm could be sorted either manually, or by using an NLP technology.

In some embodiments, a set of eligibility criteria for a single arm or scenario can be provided. A patient cohort can be defined semantically based on inclusion criteria and negation of exclusion criteria. Eligibility criteria are comprehensively categorized into simple or complex criteria based on semantic complexity. Simple criteria usually consist of a single noun phrase (menopausal), its negation (no hypertension), or a simple quantitative comparison (age>=18 years). Complex criteria typically vary in content, the use of negation, Boolean connectors, arithmetic comparison operators, temporal connectors, comparison operators, if-then constructions, or a combination of all of the above. For criteria in need of clinical judgement or more metadata support (e.g. urinalysis: no clinically significant abnormalities), they are considered underspecified. For practical purposes, the users can explicitly translate those into either single or complex criteria. By such steps, most of the eligibility criteria can be captured by terminological expressions and comparison statements.

In some aspects, the presence of complex criteria in clinical trial information can obscure a patient eligibility requirement. For example, complex criteria may use negation, or complex operational language operators, such as if-then constructions, or a combination thereof, so that one or more simple eligibility criteria may be obscured. In further aspects, criteria in clinical trial information may be obscured by complex language, so that one or more simple eligibility criteria may be obscured. Accordingly, the clinical trial matching system resolves complex or otherwise obscured eligibility criteria into single components. According to an embodiment, the clinical trial matching system may resolve complex or otherwise obscured eligibility criteria into components joined by Boolean operators.

According to an embodiment, the clinical trial matching system may convert simple criteria to standardized criteria using the CTML, including simple statements making a single assertion (e.g. bleeding caused by Warfarin) and comparison statements of the form ‘Noun Phrase+comparison operator+quantity’ (e.g. age>=18 years). In certain embodiments, the method can use a terminology system, for example, Unified Medical Language System (UMLS). In one example, simple criteria and/or simple statements can be marked up in XML format as follows:

<criterion>

<disease_or_syndrome> bleeding @ C0019080 </disease_or_syndrome>
<functional_concept> caused by @ C1314792</functional_concept>
<medication> warfarin @ C0043031</medication>

</criterion>
<criterion>

<patient_demographics> age @ C0001779 </patient_demographics>
<comparison_operator> larger than @ C0439093 </comparison_operator>
<quantity> 18 </quantity>
<temporal_concept> years @ C0439234 </temporal_concept>

</criterion>

According to an embodiment, the clinical trial matching system may convert complex criteria to standardized, and simplified, criteria using the CTML. In some embodiments, complex criteria may be transformed into simple and comparison statements. As just one example, a complex criterion may be decomposed by making implicit semantics explicit. For example, a complex criterion such as “25-45 years of age” may become (“age>=25 years” and “age<=45 years”). As another example, a complex criterion may be decomposed by making connections explicit. For example, “lung cancer, including patients who smoke,” may become (“lung cancer” OR (“lung cancer” AND “smoke”)). As another example, a complex criterion may be decomposed by separating diagnoses, conditions, and treatment explicitly. For example, “melanoma that poorly controlled by braf inhibitor,” may become (“melanoma” AND “poorly controlled melanoma” AND “took BRAF inhibitor”). As yet another example, a complex criterion may be decomposed by expanding an incomplete list. For example, “treated by Herceptin (Tykerb, Kadcyla),” may become (“treated by Herceptin” OR “treated by Tykerb” OR “treated by Kadcyla”).

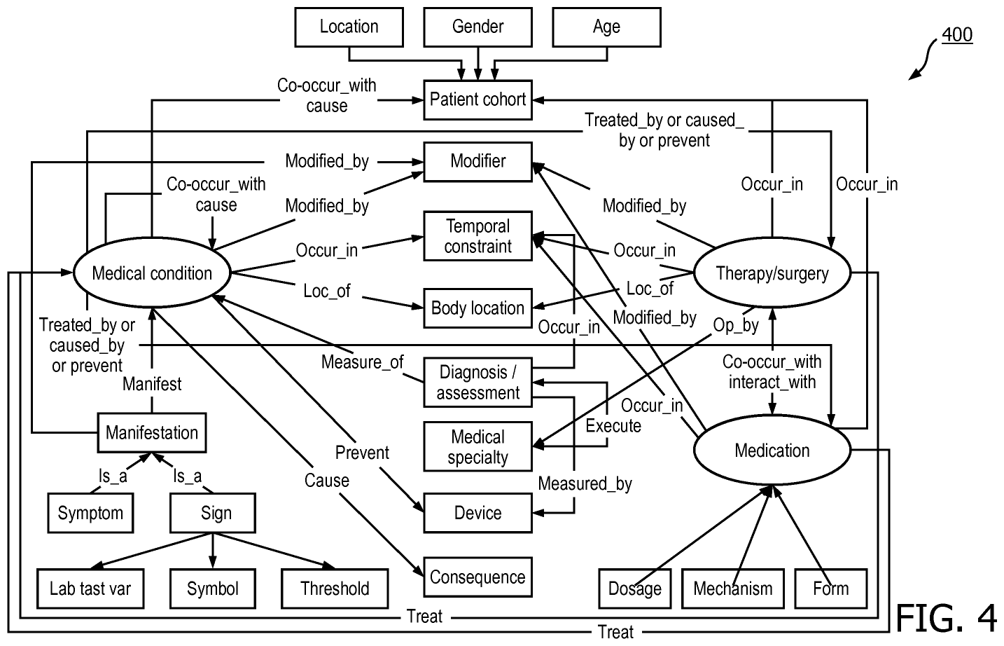

Accordingly, the system may include or comprise one or more steps for breaking down complex criteria into simple criteria. Thereafter, for each simple and comparison statement, various embodiments can provide steps for encoding simple criteria, which can be re-used recursively. When all of the simple criteria have been analysed, various embodiments can provide steps for applying Boolean connectives AND, OR, NOT, IMPLIES, or semantic/temporal/if-then connectors to stitch the individual components back into the complex one. For example, some semantic/temporal/if-then connectors are shown in FIG. 4 which comprises, in one embodiment, is a depiction of possible logic that the clinical trial matching system may utilize for analyzing and converting eligibility criteria obtained from clinical trial information. Examples of semantic/temporal/if-then connectors include “is_a,” “occur_in,” and “measured_by.”

In some aspects, the CTML may address various features when eligibility criteria are entered into a computer/processor/GUI. In one example, the CTML may provide an encoding process by addressing concept extraction and modifier extraction. In another example, the CTML may provide an encoding process by addressing formal expression logics using Boolean connectives, as well as other semantic connectors and comparison relationships, such as temporal and arithmetic connectors and comparison relationships.

At step 140 of an embodiment of a method represented in FIG. 1, the standardized patient eligibility criteria are stored in a searchable clinical trial eligibility criteria database. Each stored patient eligibility criterion is associated with the clinical trial or trials from which the criterion was extracted. Accordingly, when an eligibility criterion is identified using a query, the clinical trial associated with that eligibility criterion will also be identified. The standardized eligibility criteria and associated clinical trials may be stored in the clinical trial eligibility criteria database in or using any format. In one embodiment, the eligibility criteria and associated clinical trials are stored in a format that enables querying of the stored data, preferably in a rapid and efficient manner.

The clinical trial eligibility criteria database may be a local or remote database for use by the clinical trial matching system. For example, the clinical trial matching system may comprise the clinical trial eligibility criteria database, and/or may be in communication with a memory comprising the data structure. Accordingly, the clinical trial eligibility criteria database may be located with the clinical trial matching system or may be located remote from the clinical trial matching system, such as in cloud storage and/or other remote storage.

At step 150 of the method, the clinical trial matching system receives information about one or more patients, such as through a user interface of the system or otherwise provided, uploaded, or given to the system. For example, the clinical trial matching system may comprise a user interface configured to receive patient data, such as data entered by a clinician, a patient, or other provider. Alternatively or additionally, the clinical trial matching system may be configured to receive patient data electronically, or may be configured to receive documentation about a patient and to analyze that documentation to extract or otherwise identify patient data. This information may be stored in a database such as a patient-specific data database.

The patient information, comprising one or more patient-specific data values, provides information that may be or will be useful for determining or otherwise evaluating eligibility in a clinical trial. According to various embodiments, examples of kinds of patient-specific data include location, gender, age, medical condition, manifestation, symptom, sign, lab test results, sign symbols, sign threshold, temporal constraint, body location, diagnosis, assessment, medical specialty, device, consequence of condition, stage of condition or disease, grade of lesion or tumor, therapy, surgery, medication, dosage, mechanism of action, and/or medication form, among many other possible types or examples of patient-specific data values. In some aspects, the CTML utilized to convert eligibility criteria to a standardized format can be utilized to capture and/or convert patient-specific information in the same format as eligibility criteria and/or clinical trial information, such that the speed and accuracy of matching and recruitment of patients to clinical trials is surprisingly increased.

According to an embodiment, patient-specific data values may comprise, among other things, age, gender, gene, amino acid substitution (genomic data), cancer stage, tumor grade, and disease diagnosis. More broadly genomic information can include any gene expression, gene fusions, DNA methylation, histone modifications, and protein expression metabolomic data, among other information. Further patient information includes; patient medical conditions, manifestations, medications, therapy/surgery, and other relevant medical, quantitative self-information. According to an embodiment, clinical data may reside in Electronic Medical Record (EMR) systems, among other sources. In certain embodiments, patient data may be standardized and formalized following ISO standards, e.g. HL7/FHIR reference information model, both terminologically and logically. In additional embodiments, a VHR (Virtual Health Record) mechanism can be used to provide standard interface to heterogeneous medical record systems, which allows an additional level of translation. The structuralization can be done, for example, by user entry or fully automated parsing of clinical IT data, by for example an HL7 broker engine, among many other methods.

At step 160 of the method, the clinical trial matching system queries the clinical trial eligibility criteria database using one or more patient-specific data values. The clinical trial matching system and the clinical trial eligibility criteria database are configured to identify a stored eligibility criterion which is satisfied by a patient-specific data value. For example, the system is configured to identify an eligibility criterion as satisfied if the patient-specific data value matches the eligibility criterion, falls within or without a range specified by the eligibility criterion, and/or any other matching mechanism. The system may be configured to identify an eligibility criterion and/or identify an eligibility criterion as being satisfied when, for example, an eligibility criterion such as “age>=25 years” is met if the patient-specific data value is age=27 years. The system may be configured not to identify an eligibility criterion and/or not to identify an eligibility criterion as being satisfied when, for example, the patient-specific data value is age=21 years. Identifying an eligibility criterion as satisfied may optionally identify the clinical trial(s) associated with that eligibility criterion as being a possible clinical trial for which the patient is eligible.

According to an embodiment, a query search can fetch data and information from the translated clinical trial information, for comparing to patient-specific data and/or patient eligibility criteria to determine matching features and criteria. Suitability of a patient, and recruiting of at least one patient for the clinical trial, can involve at least one patient-specific data value matching a patient eligibility criterion of the clinical trial. In some aspects, a query can involve a plurality of factors, including any of the above patient specific data or criteria. A query module can build the query to interact with the clinical trial data base on query factors provided by the user through a user interface.

In some embodiments, a Boolean model is used for identifying matching documents and criteria, and a scoring function can be determined to calculate pertinence. For example, a query can match documents or criteria by matching Boolean combinations of other queries. The Boolean model applies the AND, OR, and NOT conditions expressed in the query to find all the documents or criteria that match. For example, the following is an example of a query that has must query, must query, and should query combined together:

```
{
“query”: {
  “bool”: {
  “must”: [
```

{ “match”: { “purpose”: “lung cancer”, “operator”:“and”}},
{ “match”: { “inclusion criteria”: “egfr”}}

]

```
  “must_not”: { “match”: { “exclusion criteria”: “pregnant” }},
  “should”: [
```

{ “match”: { “title”: “tumor” }} ]

}
}
}

This example requires that: (1) ‘lung’ and ‘cancer’ must appear in field ‘purpose’ AND (2) ‘egfr’ must appear in field ‘inclusion criteria’ AND (3) ‘pregnant’ must not appear in field ‘exclusion criteria’.

According to an embodiment, any clinical trial and/or patient data that meets the logical statements above will be a match. ‘Should’ match will not affect the bool query result, but if a document meets this criteria, it will have higher score. This process is fast, as it excludes any documents that cannot possibly match the query.

At step 170 of the method, the clinical trial matching system identifies, based on the query, one or more clinical trials for which the patient may be eligible, the clinical trial associated with one or more standardized patient eligibility criteria satisfied by the patient-specific data value(s) utilized in the query. A clinical trial may be identified when, for example, the patient-specific data satisfies one or more of the eligibility criteria for that clinical trial. According to an embodiment, a clinical trial may only be identified if a certain number of eligibility criteria are satisfied by or match the patient-specific data. According to another embodiment, the clinical trial may comprise one or more mandatory minimum eligibility criteria, each of which must be satisfied, met, or matched by the patient-specific data in order for the clinical trial to be identified. The query process may identify one clinical trial, multiple clinical trials, or no clinical trials for which the patient is eligible.

According to an embodiment, the clinical trial matching system may be configured to identify clinical trials for which the patient may be eligible, but a final determination of eligibility may be required by another system, by a human reviewer, and/or by another mechanism. For example, the system may determine that patient-specific data values satisfy one or more eligibility criteria of a clinical trial, but that clinical trial may comprise one or more eligibility criteria for which patient-specific data is not available or provided. The system may be configured to identify the clinical trial as a possibility, and may optionally flag the clinical trial or otherwise indicate that additional review or information is necessary. Many other options and embodiments are possible.

At optional step 172 of the method, the clinical trial matching system may rank two or more clinical trials identified by the query process as described or otherwise envisioned herein. According to embodiment, the clinical trial matching system may be configured to rank the identified clinical trials based at least in part on a number of standardized patient eligibility criterion satisfied by received patient-specific data values. Alternatively or additionally, the clinical trial matching system may be configured to rank the identified clinical trials based on the patient-specific data values satisfying one or more mandatory (or non-mandatory) minimum eligibility criteria of the identified clinical trial.

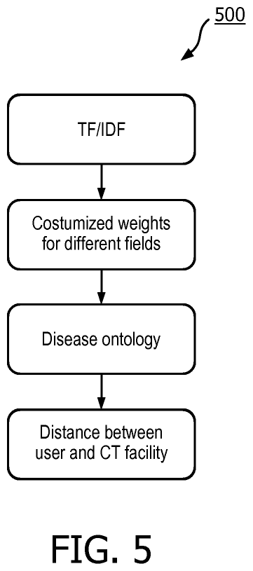

In certain embodiments, once a list of matching clinical trials and/or criteria are identified that meet the evaluation of a Boolean model, that is that the clinical trials meet the search query criteria, the clinical trials can be ranked by relevance. For example, FIG. 5 shows a block diagram of a method 500 for ranking, in accordance with an embodiment. According to an embodiment, the method comprises TF (the term frequency for term t in document d) and IDF (inverse document frequency for term t), customized weights for different fields, a disease ontology, and a factor for the distance between the user and the clinical trial facility, one or more of which may be considered when ranking identified clinical trials.

In certain embodiments, ranking can be done by utilizing Lucene's practical scoring function to calculate the score of each matched document, which is given by:

$$score ( q , d ) = {\sum }_{t ∈ q} ( tf ( t , d ) \cdot {idf ( t )}^{2} \cdot t . getBoost ( ) \cdot norm ( t , d ) ) \qquad ( Eq . 1 )$$

where score(q,d) is the relevance score of document d for query q; the summation part calculates the sum of the weights for each term t in the query q for document d; tf(t,d) is the term frequency for term t in document d (TF); idf(t) is the inverse document frequency for term t (IDF); t.getBoost( ) is the boost that has been applied to the query; and norm(t,d) is the field-length norm, combined with the index-time field-level boost. This is just one example, and many other methods for ranking and scoring are possible.

According to an embodiment, the relevance score of an entire clinical trial document may depend on the weight of each query term that appears in that document. Term frequency, inverse document frequency, and field-length norm can be used together to calculate the weight of a single term in a particular document. These may be calculated and stored at the time of indexing. Queries may consist of more than one term. Various embodiments can use a vector space model to combine the weights of multiple terms.

According to an embodiment, extra weight can be given to a field. Often, not all sections have equal importance within a clinical trial document. For example, a brief title may be more or less important than a detailed description. A section/field's weight can tuned for relevance at the time of query. Weights are assigned for each field and when calculating score a term that occurs in a field with weight 2 will get twice the score than the same term that occurs in a field with weight 1, i.e. a field with weight two is twice as important as the field with weight one. Many methods for ranking and scoring are possible.

At step 180 of the method, the clinical trial matching system may provide a report of the identified one or more clinical trials for which the patient may be eligible. The report may be provided directly to a patient, to a physician, to a clinician, and/or to any other party authorized to receive the report. Alternatively or additionally, the report may be provided electronically to another system, a patient database, a medical record management system, and/or any other recipient of electronic information.

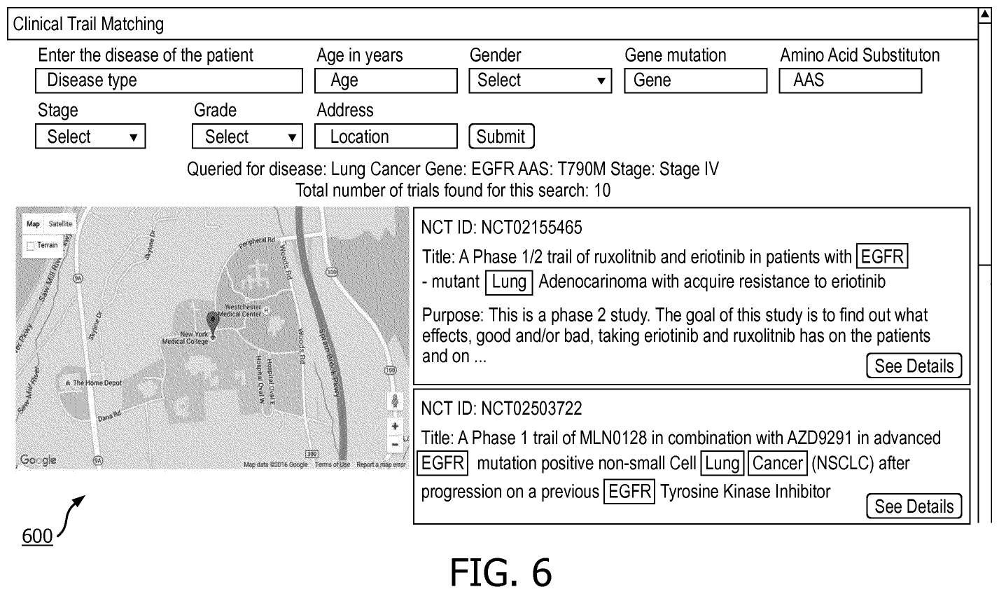

According to an embodiment, the clinical trial matching system may comprise a graphical use interface and display (GUI) for receiving and providing information. For example, the GUI may be configured to allow the user to input criteria, select further information, and view a list of pertinent clinical trials and eligibility criteria. Users may provide search queries to a web application and quickly visualize the matching trials and recruit eligible patients. Referring to FIG. 6, for example, a map can be used to recruit a patient to a trials based on geographical considerations, such as proximity to a home or treatment center, to identify clinical trials based on proximity, and/or to rank clinical trials based on proximity.

According to an embodiment, the clinical trial matching system may create a table or list of all identified clinical trials. This could be created in memory or a database, displayed on a screen or other user interface, or otherwise provided. The report or list may also comprise the eligibility criteria utilized to identify a clinical trial, as well as information about the location of the eligibility criteria within the clinical trial document. A report may be a visual display, a printed text, an email, an audible report, a transmission, and/or any other method of conveying information. The report may be provided locally or remotely, and thus the system or user interface may comprise or otherwise be connected to a communications system. For example, the system may communicate a report over a communications system such as the internet or other network. May other methods of providing, recording, reporting, or otherwise making the identified clinical trials available are possible.

According to another embodiment is a method for identifying which of a plurality of patients are eligible for a clinical trial, using a clinical trial matching system. The clinical trial matching system can be any of the systems described or otherwise envisioned herein. One or more steps of the method for identifying which of a plurality of patients are eligible for a clinical trial are similar and/or identical to the steps described in conjunction with FIG. 1 and/or method 100.

According to a further embodiment, the method comprises downloading and maintaining the most update-to-date clinical trials database(s) and identifying eligibility criteria contained therein. A dataset of clinical trial information may contain obscured and non-obscured patient eligibility criteria, which can be stored on a server. In another step, each identified eligibility criterion is encoded separately. Patient-specific data values for a plurality of patients can be input and/or received and stored by the system. According to an embodiment, the patient-specific data values are already formatted to or are converted to a standardized format, such as the structured clinical trial mark-up language described or otherwise envisioned herein. Each patient-specific data value is associated in memory with a patient, such that identification of a patient-specific data values similarly identifies the associated patient from whom the data was derived or obtained.

To identify one or more patients for a target clinical trial, eligibility criteria from the target clinical trial are extracted and standardized using the structured clinical trial mark-up language, as described or otherwise envisioned herein. The standardized eligibility criteria can then be utilized to query the patient-specific data database using any of the methods described or otherwise envisioned herein. For example, to use structuralized patient data to answer questions proposed in some eligibility criteria for patient trial matching and recruitment, each criterion may be further translated into SQL queries. SQL queries may be used in a relational database protocol to determine suitable recruitment and/or matching of a specific patient to a specific clinical trial.

The system can identify one or more patients which meet or satisfy the standardized eligibility criteria used to query the database. The identified one or more patients can be provided in a report, list, or any other method for communication.

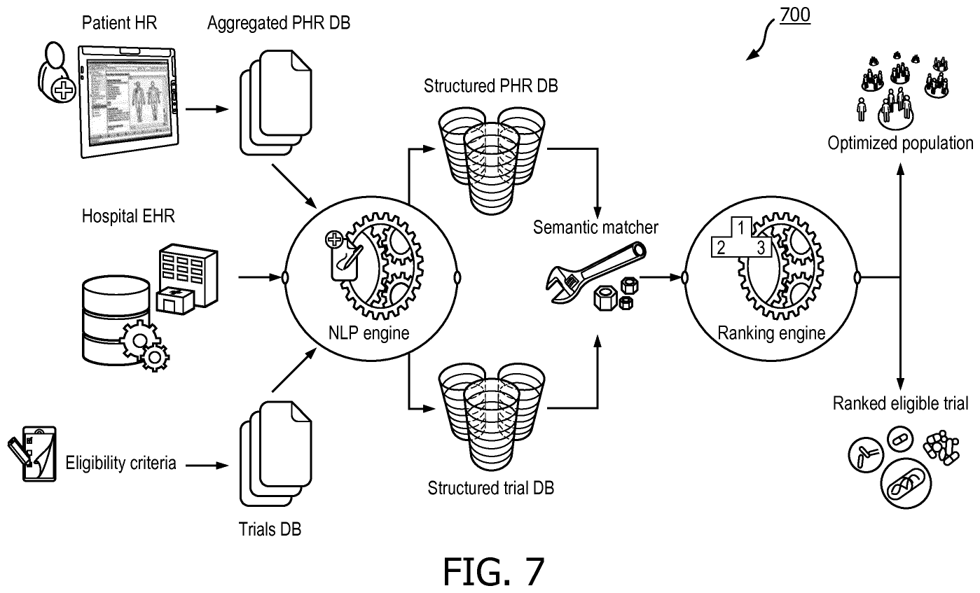

Referring to FIG. 7, in one embodiment, is a schematic representation of a method 700 for identifying one or more clinical trials for which a patient is potentially eligible, and/or for identifying one or more patients which satisfy a clinical trial. The first step, or module, includes downloading and maintaining the most update-to-date clinical trials database(s). Downloading the clinical trials database can be done before conducting a search. The clinical trials each comprise one or more eligibility criteria, which are processed by a natural language processing engine and stored in a structured clinical trial database (Structured Trial DB).

Information about patients is received by the system, such as from personal health records (PHR) and/or from electronic health records (EHR). The information is processed by a natural language processing engine and stored in a structured patient-specific data value database (Structured PHR DB).

The structured clinical trial database can be queried using patient-specific data values to identify one or more clinical trials for which a patient eligible. Similarly, the structured patient-specific data value database can be queried using eligibility criteria to identify one or more patients which are eligible for the clinical trial. The identified one or more clinical trials can be ranked to provide a ranked list of eligible clinical trials. Similarly, the identified one or more patients can be ranked and/or otherwise optimized to provide an optimized population of patients eligible for the clinical trial.

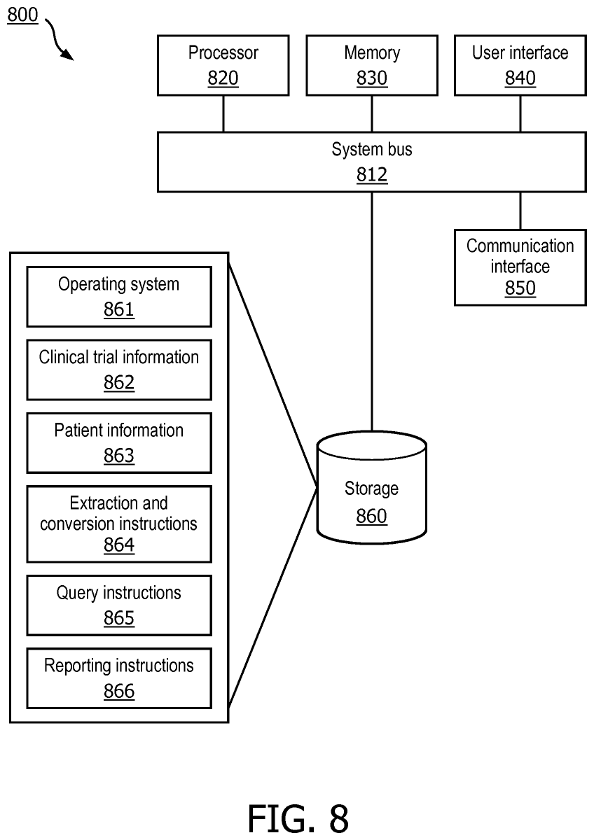

Referring to FIG. 8, in one embodiment, is a schematic representation of a clinical trial matching system 800 for identifying matching patient(s) and clinical trial(s). System 800 may be any of the systems described or otherwise envisioned herein, and may comprise any of the components described or otherwise envisioned herein.

According to an embodiment, system 800 comprises one or more of a processor 820, memory 830, user interface 840, communications interface 850, and storage 860, interconnected via one or more system buses 812. It will be understood that FIG. 8 constitutes, in some respects, an abstraction and that the actual organization of the components of the system 800 may be different and more complex than illustrated.

According to an embodiment, system 800 comprises a processor 820 capable of executing instructions stored in memory 830 or storage 860 or otherwise processing data to, for example, perform one or more steps of the method. Processor 820 may be formed of one or multiple modules. Processor 820 may take any suitable form, including but not limited to a microprocessor, microcontroller, multiple microcontrollers, circuitry, field programmable gate array (FPGA), application-specific integrated circuit (ASIC), a single processor, or plural processors.

Memory 830 can take any suitable form, including a non-volatile memory and/or RAM. The memory 830 may include various memories such as, for example L1, L2, or L3 cache or system memory. As such, the memory 830 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 800. It will be apparent that, in embodiments where the processor implements one or more of the functions described herein in hardware, the software described as corresponding to such functionality in other embodiments may be omitted.

User interface 840 may include one or more devices for enabling communication with a user. The user interface can be any device or system that allows information to be conveyed and/or received, and may include a display, a mouse, and/or a keyboard for receiving user commands In some embodiments, user interface 840 may include a command line interface or graphical user interface that may be presented to a remote terminal via communication interface 850. The user interface may be located with one or more other components of the system, or may located remote from the system and in communication via a wired and/or wireless communications network.

Communication interface 850 may include one or more devices for enabling communication with other hardware devices. For example, communication interface 850 may include a network interface card (NIC) configured to communicate according to the Ethernet protocol. Additionally, communication interface 850 may implement a TCP/IP stack for communication according to the TCP/IP protocols. Various alternative or additional hardware or configurations for communication interface 850 will be apparent.

Storage 860 may include one or more machine-readable storage media such as read-only memory (ROM), random-access memory (RAM), magnetic disk storage media, optical storage media, flash-memory devices, or similar storage media. In various embodiments, storage 860 may store instructions for execution by processor 820 or data upon which processor 820 may operate. For example, storage 860 may store an operating system 861 for controlling various operations of system 800. Storage 860 may also store clinical trial information 862 and/or patient-specific information 863.

It will be apparent that various information described as stored in storage 860 may be additionally or alternatively stored in memory 830. In this respect, memory 830 may also be considered to constitute a storage device and storage 860 may be considered a memory. Various other arrangements will be apparent. Further, memory 830 and storage 860 may both be considered to be non-transitory machine-readable media. As used herein, the term non-transitory will be understood to exclude transitory signals but to include all forms of storage, including both volatile and non-volatile memories.

While clinical trial matching system 800 is shown as including one of each described component, the various components may be duplicated in various embodiments. For example, processor 820 may include multiple microprocessors that are configured to independently execute the methods described herein or are configured to perform steps or subroutines of the methods described herein such that the multiple processors cooperate to achieve the functionality described herein. Further, where one or more components of system 800 is implemented in a cloud computing system, the various hardware components may belong to separate physical systems. For example, processor 820 may include a first processor in a first server and a second processor in a second server. Many other variations and configurations are possible.

According to an embodiment, storage 860 of clinical trial matching system 800 may store one or more algorithms and/or instructions to carry out one or more functions or steps of the methods described or otherwise envisioned herein. For example, processor 820 may comprise, among other instructions, extraction and conversion instructions 864, query instructions 865, and reporting instructions 866.

According to an embodiment, extraction and conversion instructions 864 direct the system to extract patient eligibility criteria from a clinical trial, and/or to extract patient-specific data from patient information. According to an embodiment, the extraction and conversion instructions are or comprise a language analyzer or other algorithm, such as a machine learning algorithm, configured to identify an eligibility criterion and extract or otherwise isolate or characterize the identified eligibility criterion for downstream processing or analysis by the system. According to an embodiment, the system receives information about clinical trials and stores the clinical trial information as one or more XML files, such as in the clinical trial information database 862. The clinical trial data can be structured and/or normalized using an XML parser. The XML document parser may be used to parse stored clinical trial documents and extract useful information.

The extraction and conversion instructions 864 further direct the system to convert the extracted patient eligibility criteria, and/or patient-specific data, to a standardized format using a structured clinical trial mark-up language (CTML). The CTML, which enables interoperability between one or more clinical trial documents and various patient specific clinical data, can be utilized by one or more natural language processing (NLP) tools such that the clinical trial matching system can convert unstructured clinical trial descriptions into standardized patient eligibility criteria using the CTML. According to an embodiment, the natural language processing tool can be used to translate trial information and formatted patient eligibility criteria into a series of structured data suitable for queries. Examples of NPL tools include but are not limited Stanford's Core NLP Suite, Natural language Toolkit, Apache Lucene and Solr, Apache OpenNLP, GATE, and Apache UIMA, among many other possibilities.

According to an embodiment, once the patient eligibility criteria and/or patient-specific data are converted or reformatted to a standardized format using the structured clinical trial mark-up language, the patient eligibility criteria and/or patient-specific data are stored in a database, such as clinical trial information database 862 and patient information database 863.

According to an embodiment, query instructions 865 direct the system to query the patient eligibility criteria and/or patient-specific data, such as querying clinical trial information database 862 and/or patient information database 863. For example, query instructions 865 direct the system to query the eligibility criteria in the clinical trial information database using one or more patient-specific data values. The clinical trial matching system and the clinical trial eligibility criteria database are configured to identify a stored eligibility criterion which is satisfied by a patient-specific data value. Similarly, query instructions 865 direct the system to query the patient-specific data in the patient information database using one or more clinical trial eligibility criteria. The clinical trial matching system and the patient information database are configured to identify stored patient-specific data, and the respective patient, which satisfies the one or more clinical trial eligibility criteria.

According to an embodiment, reporting instructions 866 direct the system to generate, report, and/or provide the one or more identified clinical trials for which a patient is eligible. Similarly, the reporting instructions 866 direct the system to generate, report, and/or provide the one or more patients which are eligible for a clinical trial. For example, the system may create a table or list of all identified clinical trials and/or identified patients. This could be created in memory or a database, displayed on a screen or other user interface, or otherwise provided. A report may be a visual display, a printed text, an email, an audible report, a transmission, and/or any other method of conveying information. The report may be provided locally or remotely, and thus the system or user interface may comprise or otherwise be connected to a communications system.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of;” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of;” “only one of,” or “exactly one of”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

1. A computer-implemented method for matching a patient with a clinical trial using a clinical trial matching system, comprising:
receiving a dataset comprising information about one or more clinical trials, the information comprising one or more patient eligibility criterion for each of the one or more clinical trials;
extracting, by a processor of the system, the one or more patient eligibility criterion from each of the one or more clinical trials;
converting, by the processor, each of the extracted patient eligibility criterion to a standardized patient eligibility criterion using a structured clinical trial mark-up language;
storing the standardized patient eligibility criterion in a searchable clinical trial eligibility criteria database, each of the standardized patient eligibility criterion associated with at least one of the one or more clinical trials;
receiving one or more patient-specific data values about a patient;
querying, by the processor, the clinical trial eligibility criteria database using the received one or more patient-specific data values to identify one or more standardized patient eligibility criterion satisfied by a received patient-specific data value;
identifying at least one of the one or more clinical trials, the at least one clinical trial associated with the one or more standardized patient eligibility criterion satisfied by a received patient-specific data value; and
providing a report of the identification of the at least one clinical trial.

2. The method of claim 1, further comprising the step of ranking two or more identified clinical trials, wherein the ranking is based at least in part on a number of standardized patient eligibility criterion satisfied by received patient-specific data values, and wherein the report comprises information about the ranking of the two or more identified clinical trials.

3. The method of claim 1, wherein the report is provided via a user interface of the system.

4. The method of claim 1, the dataset comprising information about one or more clinical trials is comprised of information from a plurality of sources.

5. The method of claim 1, wherein the step of converting the extracted patient eligibility criterion to a standardized patient eligibility criterion comprises a machine learning algorithm.

6. The method of claim 1, wherein the step of converting the extracted patient eligibility criterion to a standardized patient eligibility criterion comprises resolving a complex eligibility criterion into one or more simple eligibility criteria.

7. The method of claim 6, wherein the one or more simple eligibility criteria are joined by one or more Boolean operators.

8. The method of claim 1, wherein the one or more patient eligibility criterion comprise inclusion criteria and exclusion criteria.

9. The method of claim 1, wherein the one or more patient-specific data values are obtained from a patient medical record.

10. A system for matching a patient with a clinical trial, comprising:
a clinical trial eligibility criteria database comprising information about a plurality of clinical trials, each of the plurality of clinical trials comprising one or more patient eligibility criterion; and
a processor configured to: (i) extract the one or more patient eligibility criterion from each of the one or more clinical trials; (ii) convert each of the extracted patient eligibility criterion to a standardized patient eligibility criterion using a structured clinical trial mark-up language; (iii) store the standardized patient eligibility criterion in the clinical trial eligibility criteria database, each of the standardized patient eligibility criterion associated with at least one of the one or more clinical trials; (iv) receive one or more patient-specific data values about a patient; (v) query the clinical trial eligibility criteria database using the received one or more patient-specific data values to identify one or more standardized patient eligibility criterion satisfied by a received patient-specific data value; (vi) identify at least one of the one or more clinical trials, the at least one clinical trial associated with the one or more standardized patient eligibility criterion satisfied by a received patient-specific data value; and (vii) generate a report of the identification of the at least one clinical trial.

11. The system of claim 10, wherein the processor is further configured to rank two or more identified clinical trials, wherein the ranking is based at least in part on a number of standardized patient eligibility criterion satisfied by received patient-specific data values, and wherein the report comprises information about the ranking of the two or more identified clinical trials.

12. The system of claim 10, wherein the system further comprises a user interface, and the report is provided via the user interface.

13. The system of claim 10, wherein the processor is configured to resolve complex eligibility criteria into one or more simple eligibility criteria.

14. The system of claim 10, further comprising a patient information database, the patient information database comprising one or more patient-specific data values.

15. A computer-implemented method for matching a patient with a clinical trial using a clinical trial matching system, comprising:
receiving a dataset comprising information about one or more clinical trials, the information comprising one or more patient eligibility criterion for each of the one or more clinical trials;
extracting, by a processor of the system, the one or more patient eligibility criterion from each of the one or more clinical trials;
converting by the processor, each of the extracted patient eligibility criterion to a standardized patient eligibility criterion using a structured clinical trial mark-up language;
receiving one or more patient-specific data values about a patient, and storing the patient-specific data values in a patient information database;
querying, by the processor, the patient information database using the standardized one or more patient eligibility criterion to identify one or more patients eligible for a clinical trial;
identifying at least one of the patients, the at least one patient associated with a patient-specific data value satisfying a standardized patient eligibility criterion used to query the patient information database; and
providing a report of the identification of the at least one patient.
